(19) United States
(12) Patent Application Publication
(10) Pub. No.: US 2026/0108888 A1
(43) Pub. Date: Apr. 23, 2026

(54) DEVICE AND METHOD FOR SINGLE EXOSOME PROTEOFORM ANALYSIS

(71) Applicant: University of Georgia Research Foundation, Inc., Athens, GA (US)

(72) Inventors: Yang Liu, Bogart, GA (US); Luyao Zhao, Athens, GA (US); Yiping Zhao, Bogart, GA (US)

(21) Appl. No.: 19/367,093

(22) Filed: Oct. 23, 2025

Related U.S. Application Data
(60) Provisional application No. 63/710,646, filed on Oct. 23, 2024.

(51) Int. Cl. B01L 3/00; G01N 21/65; G01N 33/532

(52) U.S. Cl. CPC B01L 3/502761; B01L 3/502784; G01N 21/658; G01N 33/532; B01L 2200/0647; B01L 2300/0645; B01L 2300/0819; B01L 2300/0893

(57) ABSTRACT

In an aspect, the present discourse provides for a microfluidic device and system that can be used to prepare droplets that include an exosome, separate the droplets, and analyze components of the exosome.

### CLAIM OF PRIORITY TO RELATED APPLICATION

This application claims priority to co-pending U.S. provisional application entitled “DEVICE AND METHOD FOR SINGLE EXOSOME PROTEOFORM ANALYSIS” having Serial No.: 63/710,646 filed on Oct. 23, 2024, which is entirely incorporated herein by reference.

### BACKGROUND

Exosomes are nanosized extracellular vesicles (EVs) with sizes between 50-150 nm. They are secreted by cells and play a role in physiological and pathological processes. Exosomes have unique advantages in disease diagnosis and treatment compared to synthetic carriers like liposomes and nanoparticles because they carry various bioactive substances including nucleic acids, proteins, and lipids. Specifically, two types of proteins, such as transmembrane or GPI-anchored proteins associated with plasma membrane and/or endosomes, and cytosolic proteins are recovered in extracellular vesicles, pivoting to their biogenesis, specificity, and intercellular functions. There is a need to accurately measure these materials.

### SUMMARY

In accordance with the purpose(s) of this disclosure, as embodied and broadly described herein, the disclosure, in various aspects, provides for a microfluidic device and system that can be used to prepare droplets that include an exosome, separate the droplets, and analyze components of the exosome.

Embodiments of the present disclosure provide for a microfluidic system, comprising: a first structure including a first channel configured to receive a plurality of exosomes, a second channel configured to receive a hydrogel precursor, where the second channel merges with the first channel to form a third channel, wherein the exosomes are within the hydrogel precursor in the third channel, and a fourth channel intersecting the third channel, wherein the system is configured to form a plurality of droplets in the third channel after the intersection with the fourth channel; and a second structure having a first plurality of microwells, wherein each microwell is configured to receive a single droplet, wherein each microwell includes a top layer, wherein the top layer is a gel layer having a top side and a bottom side, wherein the gel layer receives the droplet on the top side.

Embodiments of the present disclosure provide for a method, comprising: loading a plurality of exosomes into a respective plurality of droplets, such that each exosome of the plurality of exosomes is contained within a respective droplet of the plurality of droplets; loading each droplet of the plurality of droplets into a respective microwell of a plurality of microwells in a microfluidic device; lysing the plurality of exosomes within the plurality of droplets; and applying an electric field to release a plurality of proteins from each exosome to a gel layer.

Embodiments of the present disclosure provide for a system, comprising: a microfluidic device having a first structure and a second structure, wherein the first structure comprises: a first structure having a first channel configured to receive a plurality of exosomes, a second channel configured to receive a hydrogel precursor, where the second channel merges with the first channel to form a third channel, wherein the exosomes are within the hydrogel precursor in the third channel, and a fourth channel intersecting the third channel configured to cross-flow a fluid against the plurality of exosomes in the hydrogel precursor to form the droplet, wherein the third channel is in fluidic communication with the plurality of microwells; wherein the second structure includes a plurality of microwells, each microwell of the plurality of microwells configured to receive a single droplet; and an electrophoresis machine configured to apply an electric current to the plurality of microwells.

Other systems, methods, devices, features, and advantages of the devices and methods will be or become apparent to one with skill in the art upon examination of the following drawings and detailed description. It is intended that all such additional systems, methods, devices, features, and advantages be included within this description, be within the scope of the present disclosure, and be protected by the accompanying claims.

### BRIEF DESCRIPTION OF THE DRAWINGS

Further aspects of the present disclosure will be more readily appreciated upon review of the detailed description of its various embodiments, described below, when taken in conjunction with the accompanying drawings. The components in the drawings are not necessarily to scale, emphasis instead being placed upon clearly illustrating the principles of the present disclosure. Moreover, in the drawings, like reference numerals designate corresponding parts throughout the several views.

FIG. 1 is a schematic illustration of the microwell device. The exosomes can be loaded in microwells individually by microdroplet. Fluorophore signals can be promoted by immunoprobing.

FIGS. 2A-B are illustrations showing loading of RhoB. FIG. 2A shows RhoB loading on gel with diffusion down to AgNR substrate. FIG. 2B shows direct loading of RhoB on AgNR substrate and covered with gel.

FIG. 3 shows a comparison of loading RhoB on gel vs. loading gel on RhoB. The peaks signature to RhoB are designated with arrows.

FIGS. 4A-B show SERS spectra for RhoB diffusion through gel. FIG. 4A is the SERS spectra collected with 785 nm laser after a given amount of time for 0.01 mg/mL RhoB diffusion through gel. All peaks are signatures of RhoB. The peaks with asterisks were analyzed for intensity. FIG. 4B shows peak intensity analysis of time-dependent spectra.

FIGS. 5A-B show COMSOL simulations of BSA traveling through PA-gel towards SERS substrate at the bottom of the gel via passive diffusion (FIG. 5A) and electrophoresis (FIG. 5B). Microwells have diameter and depth of 40 μm and gel has dimensions of 400 μm by 80 μm.

FIG. 6 is a schematic illustration showing an example methodology for single exosome proteoform analysis.

FIGS. 7A-D are images showing the setup for the single exosome project. FIG. 7A shows the SERS substrate with PAGE-gel Microwell Array. FIG. 7B shows the droplet generation device. FIG. 7C shows the 2D Electrophoresis setup. FIG. 7D shows the 3D Electrophoresis setup.

FIG. 8 is an illustration depicting the exosomes being captured into droplets and then being loaded into the gel microwell array. Once in the microwell device the proteins in the exosomes are subject to separation by gel electrophoresis.

FIG. 9 shows the wafer device for droplet formation and formed droplets.

FIG. 10 shows a view of the microwells. The microwells are 7 μm in diameter.

FIG. 11 shows a view of the microwells with the gel beads loaded into the microwells.

### DETAILED DESCRIPTION

Before the present disclosure is described in greater detail, it is to be understood that this disclosure is not limited to particular embodiments described, as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit (unless the context clearly dictates otherwise), between the upper and lower limit of that range, and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, chemistry, biological, microfluidic techniques and the like, which are within the skill of the art. Such techniques are explained fully in the literature. The following examples are put forth to provide those of ordinary skill in the art with a complete disclosure and description of how to perform the methods and use the compositions and compounds disclosed and claimed herein. Efforts have been made to ensure accuracy with respect to numbers (e.g., amounts, measurements, etc.), but some errors and deviations should be accounted for.

Before the embodiments of the present disclosure are described in detail, it is to be understood that, unless otherwise indicated, the present disclosure is not limited to particular materials, machines, computing processes, or the like, as such can vary. It is also to be understood that the terminology used herein is for purposes of describing particular embodiments only and is not intended to be limiting. It is also possible in the present disclosure that steps can be executed in different sequence where this is logically possible.

It should be noted that ratios, amounts, and other numerical data can be expressed herein in a range format. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to about 5%” should be interpreted to include not only the explicitly recited values of about 0.1% to about 5%, but also include individual values (e.g., 1%, 2%, 3%, and 4%) and the sub-ranges (e.g., 0.5%, 1.1%, 2.2%, 3.3%, and 4.4%) within the indicated range. Where the stated range includes one or both of the limits, ranges excluding either or both of those included limits are also included in the disclosure, e.g., the phrase “x to y” includes the range from ‘x’ to ‘y’ as well as the range greater than ‘x’ and less than ‘y’. The range can also be expressed as an upper limit, e.g., ‘about x, y, z, or less’ and should be interpreted to include the specific ranges of ‘about x’, ‘about y’, and ‘about z’ as well as the ranges of ‘less than x’, less than y', and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y', and ‘greater than z’. In some embodiments, the term “about” can include traditional rounding according to significant figures of the numerical value. In addition, the phrase “about ‘x’ to ‘y’”, where ‘x’ and ‘y’ are numerical values, includes “about ‘x’ to about ‘y’”.

### Discussion

Extracellular vesicles (EVs), particularly exosomes, are pivotal in mediating intercellular communication, carrying an array of biomolecules including proteins, nucleic acids, and lipids from their cells of origin to distant cellular destinations. Exosomes, typically ranging from about 30-150 nm in diameter, are formed through endosomal pathways and secreted across various bodily fluids, making them accessible markers for numerous diseases. Despite their promising utility in clinical diagnostics, the heterogeneity and overlap in protein profiles among EV subtypes underscore the necessity for standardization in exosome isolation and analysis techniques.

The characterization and analysis of exosomes have evolved through various methodologies. Nanoparticle Tracking Analysis (NTA) and Dynamic Light Scattering (DLS) are commonly used in detecting particle size and distribution data. Nanoparticle Tracking Analysis (NTA) shines in its ability to concurrently assess particle size and concentration with a resolution sensitive enough to detect the subtle heterogeneity within exosome populations, although its accuracy may diminish in polydispersed samples due to signal overlap. Dynamic Light Scattering (DLS), while offering rapid and non-invasive size distribution analysis with minimal sample preparation, may preferentially highlight larger particles, potentially skewing size distribution analyses in mixed samples. Electron Microscopy (EM), encompassing both transmission and scanning variants, provides unparalleled morphological detail at the nanoscale, despite its intensive sample preparation requirements and the need for expert interpretation.

Mass spectrometry is currently capable of elucidating exosome proteome structure and composition. However, its application at the level of individual exosomes is fraught with technical challenges, mainly due to the limited sensitivity in detecting low-abundance proteins within individual vesicles. On the other hand, flow cytometry has been used to detect and quantify individual exosomes using its high-throughput analysis capabilities, but flow cytometry is still limited by cross-reactivity issues and lacks specificity in identifying protein isoforms.

Western blotting serves as a stalwart tool for the specific detection of proteins within exosomes, vital for confirming the presence of exosomal markers, though its semi-quantitative nature and intensive sample demands might limit its application. Mass Spectrometry (MS)-based proteomic analysis, in contrast, uncovers a rich landscape of proteins, lipids, and metabolites, providing a comprehensive molecular profile with high resolution, despite the requisite sophisticated equipment and analytical expertise. Flow cytometry, adept at rapid and high-throughput analysis of surface markers, brings the potential for sorting exosomes based on specific proteins, though its utility is tempered by size detection limits that may exclude smaller vesicles and by sample preparation challenges that can affect vesicle integrity and aggregation. According to current technology, there is a lack of standardized, high sensitivity, low-limit of detection assays for individual exosomal proteins. Thus, it is necessary to develop a technique that can analyze exosome protein heterogeneity at a single level. The present disclosure introduces a breakthrough technique for high-resolution, label-free detection of proteoforms in single exosomes that utilizes the precision of single-cell Western Blot (seWB) to achieve individual vesicle resolution in protein heterogeneity analysis.

The present disclosure applies seWB technology to exosome studies, bridging microfluidics with the molecular specificity of western blotting. By encapsulating individual exosomes within microdroplets, employing polyacrylamide gel electrophoresis (PAGE), and utilizing targeted immunolabeling, label-free SERS imaging, proteins in individual exosomes can be identified based on molecular weight.

The present disclosure provides for a microfluidic device integrating silicon chips with PAGE gels specifically for encapsulating and analyzing individual exosomes. Starting with exosomes isolated using commercial kits or ultrahigh centrifugation or any separation methods, the process of microdroplet generation and microwell array loading is refined to achieve high precision and repeatability. The present disclosure can advance biomarker discovery and enhance the potency of exosome-based diagnostics and therapeutics. Accordingly, various embodiments of the present disclosure are directed to systems and methods for single exosome proteoform analytics.

In the following discussion, a general description of the device and system and its components is provided, followed by a discussion of the operation of the same. Although the following discussion provides illustrative examples of the operation of various components of the present disclosure, the use of the following illustrative examples does not exclude other implementations that are consistent with the principles disclosed by the following illustrative examples.

In an aspect, the present discourse provides for microfluidic devices and systems that can be used to prepare droplets that include an exosome, separate the droplets, and analyze components of the exosome. In an aspect, the present discourse provides for a microfluidic system and device that includes a first structure and a second structure. The first structure can be used to prepare the droplets to include an exosome, while the second structure receives the droplets and then is configured to analyze the components (e.g., proteins) of the exosome. The first structure can be used to prepare the droplets. The first structure includes a first channel configured to receive a plurality of exosomes. The first structure includes a second channel configured to receive a hydrogel precursor where the second channel merges with the first channel to form a third channel. For example, the first channel and the second channel merge at an angle forming a “Y” shape to form the third channel. The exosomes and the hydrogel precursor will meet and mix in the third channel. A fourth channel intersects the third channel to introduce a fluid. For example, the fourth channel can intercept the third channel at opposing sides of the third channel in a substantially perpendicular or perpendicular manner. In an aspect, the positioning of the fourth channel can be designed so that the fluid (e.g., an oil) flowing out of the fourth channel forms around (e.g., like a bubble of the oil around each exosome) each exosome to form the droplet. In an aspect, the droplet can have a diameter that is about ⅔ the diameter of the microwell. In an aspect, the droplet can have a droplet diameter of about 1-10 μm (e.g., volume of about 0.5-524 fL). In an aspect, the channel after the intersection of the third channel and fourth channel can optionally be referred to as the fifth channel, which is where the droplets are flowed. In an aspect, the width of the third channel after the intersection with the fourth channel can be increased.

The hydrogel precursor can be a material(s) that upon exposure to UV light forms a hydrogel (e.g., a material and a photoinitiator). The hydrogel can be one that is compatible with exosomes. The hydrogel can be one that when mixed with the fluid (e.g., oil) can from droplets. The hydrogel can be polymer based hydrogels such as poly(ethylene glycol) based hydrogels polyvinyl alcohol hydrogels, gelatine hydrogels, alginate hydrogels, peptide based hydrogels.

In an aspect, the third channel is in fluidic communication with the second structure, where the second structure includes a plurality of microwells. Each microwell is configured to receive a single droplet. The microfluidic device can have 5,000-500,000 microwells. In an aspect, the microwell can have a spacing in the y-direction of about 100 μm×1000 μm, where the microwell spacing in the x-direction is about 20 μm×200 μm. In an aspect, each microwell has a diameter that is about 1.5× the diameter of a single droplet. In an aspect, each microwell can have a diameter of about 1.5 to 15 μm, about 6 to 10 μm, or about 8 μm. In an aspect, each microwell can have a height (depth) of about 2 to 20 μm, about 6 to 14 μm, or about 8 to 12 μm, or about 10 μm.

In an aspect, the second structure can include a gel layer and/or a Raman substrate layer. The gel layer is positioned on top of the Raman substrate layer, when the Raman substrate layer is present. The gel layer receives the droplet on a first side and the Raman substrate layer is on the side opposite the first side of the gel layer. Once lysed, proteins from the exosome can be separated by molecular weight (in the x- and y-directions) and can then be pulled (in the z-direction) into the Raman substrate layer using electrophoresis. The gel layer and/or a Raman substrate layer are described in more detail herein.

In an aspect, the first channel can have a length of about 10,000 to 30,000 μm, a width (or diameter) of about 7 to 30 μm. In an aspect, the second channel can have a length of about 10,000 to 30,000 μm, a width (or diameter) of about 7 to 30 μm. In an aspect, the third channel can have a length of about 500 to 3000 μm, a width (or diameter) of about 20 to 150 μm. In an aspect, the fourth channel can have a length of about 10,000 to 45,000 μm, a width (or diameter) of about 20 to 150 μm. In an aspect, each channel can have a height of about 1 to 10 μm. If the channel after the intersection of the third and fourth channels is referred to as the fifth channel, the fifth channel can have the same or similar dimensions as the third channel. In an aspect, the third channel, after the intersection with the fourth channel can have a diameter that is 110 to 200% the width of the section of the third channel prior to the intersection or in another embodiment the diameter can be 50 to 90% the width of the section of the third channel prior to the intersection.

The present disclosure also includes a method of capturing and analyzing exosome and components of the exosome. In an aspect, the method includes loading a plurality of exosomes into a respective plurality of droplets, such that each exosome of the plurality of exosomes (e.g., 0 to 10 or 1 to 10) is contained within a respective droplet of the plurality of droplets.

In an aspect, the loading step includes inserting a plurality of exosomes into a hydrogel precursor, flowing the hydrogel precursor and plurality of exosomes into a microchannel (e.g., third microchannel), and intersecting the flowing of the hydrogel precursor with a crossflow of a fluid to form the droplets, where the droplets can include an exosome. In an aspect, the fluid is an oil, mineral oil, and HFE 7500 oil. The fluid can be a combination of an oil and a surfactant (e.g., Span 80 or 008-fluorosurfactant).

The method includes loading each droplet of the plurality of droplets into a respective microwell of a plurality of microwells in the microfluidic device. Optionally, the droplets can be converted into gel droplets using UV light. Next, the plurality of exosomes within the plurality of droplets are lysed to release a plurality of proteins from each exosome to a gel layer. Then an electric field is applied to the gel layer to conduct 2D planar (XY) electrophoresis to separate protein based on molecular weight. Then the electric field in the 3D (in the z direction) can be used to transfer the protein from gel substrate to the SERS substrate (when SERS detection is to be conducted). In an aspect, for immunolabeling, only the 2D separation using electrophoresis is necessary. In particular, the electric field applied to the gel layer causes the plurality of proteins to migrate towards a positive electrode on the microfluidic device.

The method optionally includes applying an ultraviolet (UV) light to the gel layer. Applying the UV light fixes the plurality of proteins in place within the gel layer. The gel layer can be made of polyacrylamide gel (e.g., about 7 to 15% crosslinking) or agarose gel. In an aspect, gel strengthening agents can be added to strengthen the gel. The gel layer can have a thickness of about 10 to 200 μm. When the SERS detection is used, applying UV light is not necessary.

In an aspect, antibodies can optionally be added to each gel layer when immunolabeling is used, where the antibodies bind to the plurality of proteins within each gel layer. The antibody can be added after the gel layer is exposed to UV light. The antibody can include exosome related antibodies (e.g., anti-CD9, -CD63, -CD81, -HSP70, -TSG101, as well as other disease related antibodies: -PD-L1, -EpCAM, -Vimentin). In an aspect, the antibodies can include antibodies with fluorophore-conjugates or primary antibodies (e.g., antibody against vimentin and EpCAM) and fluorophore-conjugated secondary antibodies (e.g., Alexa Fluor 488 or 555 donkey anti-goat IgG). The antibody can include exosome related antibodies (e.g., anti -CD9, -CD63, -CD81, -HSP70, -TSG101) other disease related antibodies: -PD-L1, -EpCAM, -Vimentin). When SERS detection is used, no antibodies are needed during the process.

In an aspect, the method includes transferring the plurality of proteins from the gel layer to a Raman substrate layer of the microfluidic device by conducting 3d electrophoresis (in the z direction). In other words, electrophoresis can be performed to separate the proteins in the x-and y-direction using electrophoresis and then electrophoresis can be performed again to transport the proteins in the z-direction to the Raman substrate layer (e.g., the z-plane is perpendicular to the plane of the Raman substate layer). The Raman substrate layer can be made of silver or gold or copper nanorods. The Raman substrate layer can have a thickness of about 200 to 1500 nm. In an aspect, one or more protein signals can be detected from the plurality of proteins using Raman spectroscopy.

In an aspect, the system that includes the microfluidic device (e.g., the first structure and the second structure) as described above and herein. In an aspect, the system includes a plurality of microwells, where each microwell of the plurality of microwells is configured to receive a single droplet from the third channel (or optionally the fifth channel is this phrase is used). The system includes an electrophoresis machine configured to apply an electric current to the plurality of microwells. In an aspect, the electrophoresis machine can function to separate proteins in the x-and y-direction and also optionally transfer the proteins in the z-direction to the Raman substrate layer. In an aspect, the system also optionally includes a device to produce an ultraviolet (UV) light that is directed to the plurality of microwells to convert the droplets to gel droplets and/or directed to the gel layer. In an aspect, the system optionally includes a Raman spectrometer when SERS analysis is performed. The system can operate in a manner consistent with the description of the method(s) provided above and herein.

The following provides additional details in reference to the figures. Provided is a microfluidic device 100 (or system) which can include a first structure and a second structure.

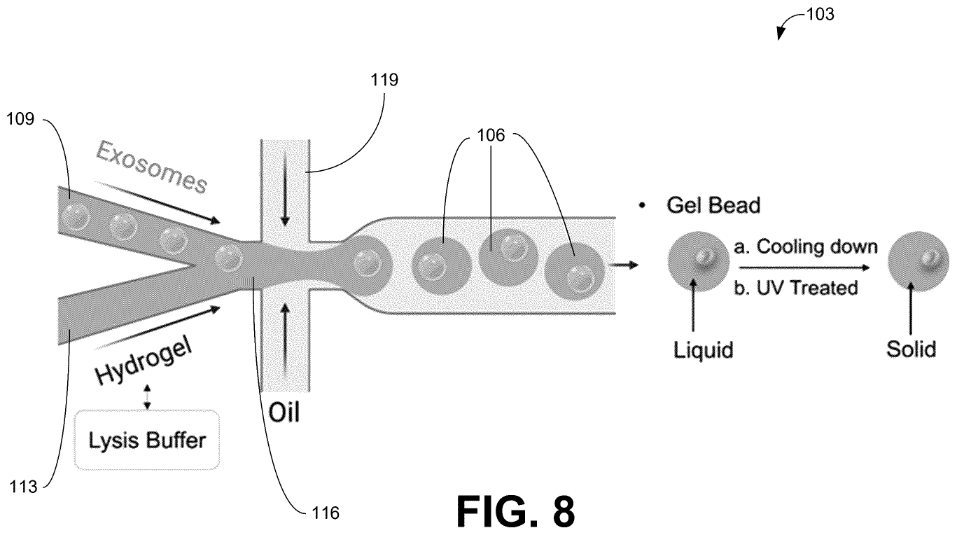

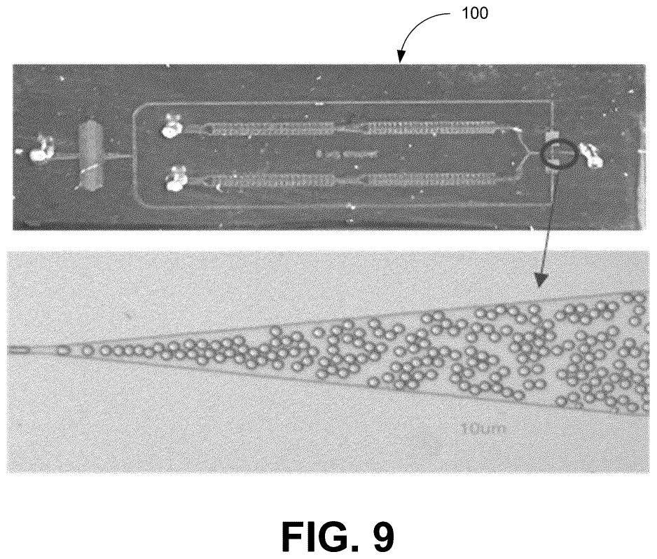

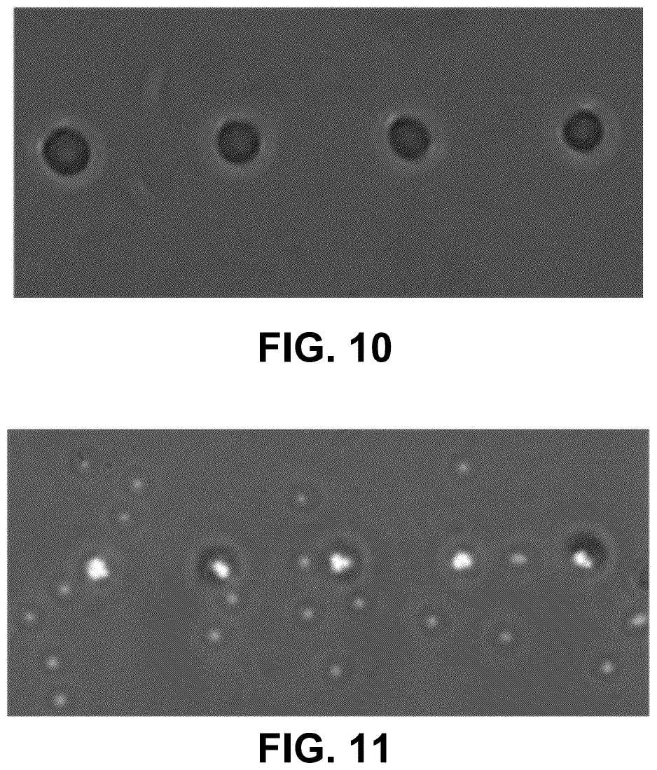

An example of the microfluidic device 100 is shown in FIG. 9. In some examples, the microfluidic device 100 has a first structure 103 that is configured to generate droplets 106 which contain exosomes. An example of the first structure 103 is shown in FIG. 8. The first structure 103 of the microfluidic device 100 can include a first channel 109 configured to receive a plurality of exosomes and a second channel 113 configured to receive a hydrogel precursor. The second channel 113 can be configured to merge with the first channel 109 to form a third channel 116. In addition, the first structure 103 can include a fourth channel 119 intersecting (e.g. perpendicularly) the third channel 116. According to various embodiments, the fourth channel 119 is configured to cross-flow a fluid against the plurality of exosomes in the hydrogel precursor. When in operation, the exosomes can flow through the first channel 109 and intersect with the hydrogel precursor in the second channel 113. Once in the third channel 116, the exosomes and the hydrogel precursor are mixed and the exosome become suspended in the hydrogel precursor. As the exosome and hydrogel mixture passes through the intersection with the fourth channel 119, the oil or other hydrophobic liquid can cause a separation of the hydrogel stream and create droplets 106. Each droplet 106 can contain an exosome. In some examples, the first structure 103 comprises poly(dimethyl siloxane) (PDMS).

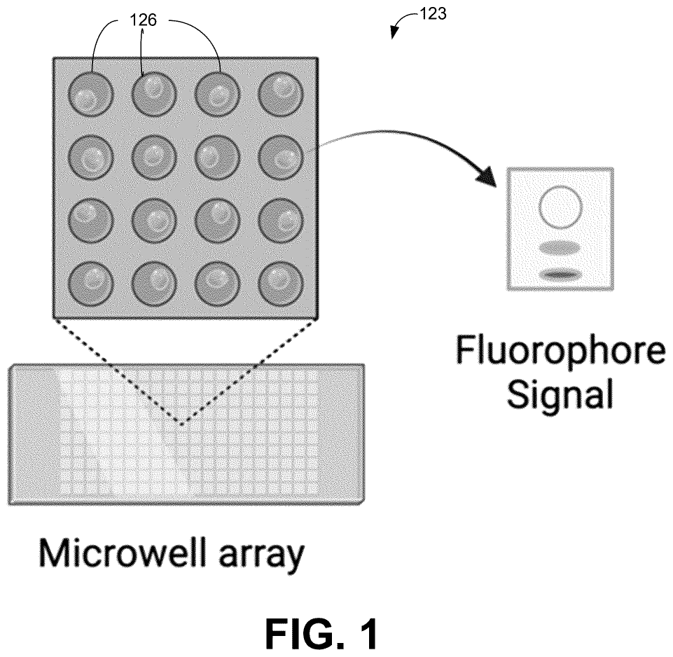

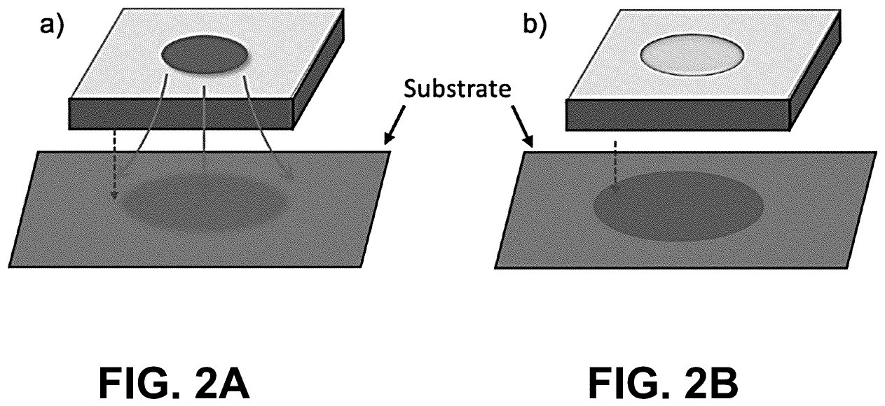

The microfluidic device 100 can include a second structure 123. The second structure 123 can be configured to facilitate electrophoresis, such as polyacrylamide gel electrophoresis (PAGE). An example of the second structure 123 is shown in FIG. 1. The second structure 123 can include a plurality of microwells 126. Each microwell 126 of the plurality of microwells 126 can be configured to receive a single droplet 106. In some examples, each microwell 126 can receive a single droplet 106 from the first structure 103 of the microfluidic device 100. In some examples, the droplets 106 can be transferred from the first structure 103 to the microwells 126 of the second structure 123. In some examples, the second structure 123 comprises a gel layer. The second structure 123 can facilitate the lysis of the exosomes as well as electrophoresis (in the x- and y-direction as well as in the z-direction depending upon the type of protein analysis) and optionally ultraviolet (UV) light exposure of the gel layer.

According to some examples, the second structure can include a Raman substrate layer 129. The proteins can be transferred from the plurality of microwells 126 of the second structure 123 to the Raman substrate layer 129 by conducting 3D electrophoresis (e.g., in the z-direction). In particular, the proteins will be released first by EV lysis, then electromigrate into the neighboring PA-Gel layer via planar electrophoresis. This step will separate proteins based on MW after that, a 3D electrophoresis transfers the protein from the gel (top layer) to the Raman substrate (bottom layer)

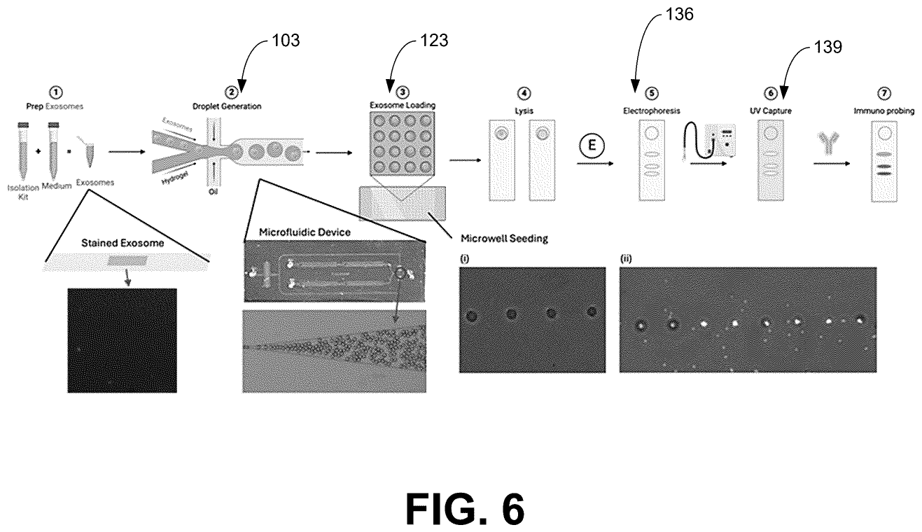

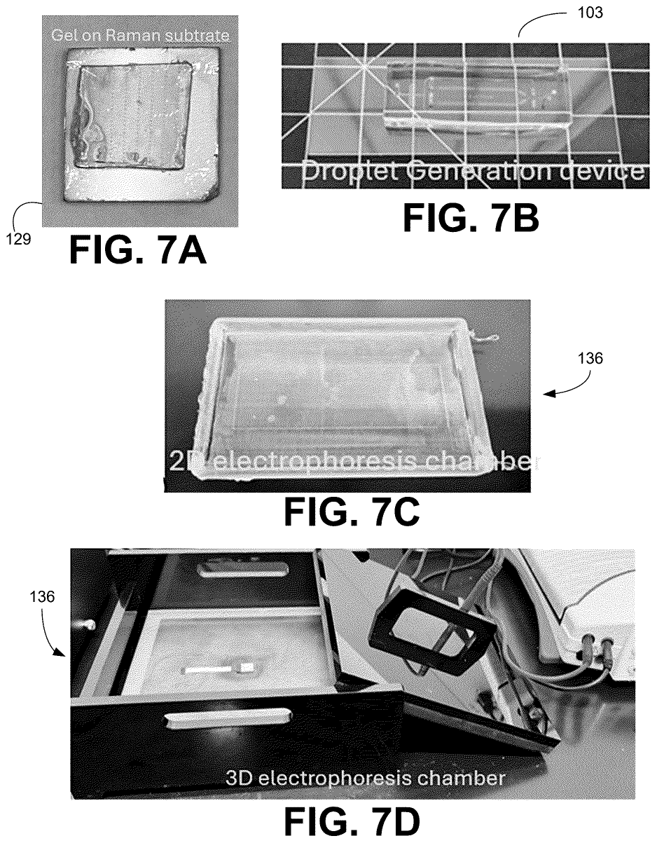

The present disclosure also provides a system that includes the microfluidic device 100 described herein. The system can also include an electrophoresis machine 136 as shown in FIGS. 6, 7C, and 12. The electrophoresis machine 136 can be configured to apply an electric current to the plurality of microwells 126. In an aspect, the system can optionally include an ultraviolet (UV) light 139 directed to the plurality of microwells 126 to form gel droplets for immunolabeling. Also, the system can optionally direct UV light toward the gel layer in the microwells to fix the proteins in place in the gel layer. In an aspect, the system can optionally include a Raman spectrometer for SERS analysis.

A method is provided having a first step of loading a plurality of exosomes into a respective plurality of droplets 106 such that each exosome of the plurality of exosomes is contained within a respective droplet 106 of the plurality of droplets 106. In some examples, this method can be performed in one microfluidic device 100 where the first step is conducted in a first structure 103 of the microfluidic device 100. As described above, loading the plurality of exosomes into the plurality of droplets 106 can further include the steps of inserting a plurality of exosomes into a hydrogel precursor by flowing and mixing a hydrogel precursor and the plurality of exosomes. In some examples, this can be accomplished by flowing the exosomes through the first channel 109 and the hydrogel precursor through the second channel 113, and intersecting the two streams to form and exosome-hydrogel precursor mixture. Next, the hydrogel precursor and plurality of exosomes can be flowed into a microchannel (e.g., the third channel 116), and the flow of the exosome-hydrogel precursor mixture can be intersected with a crossflow of fluid (e.g., oil) from the fourth channel 119 to form droplets 106.

After the exosomes have been loaded into the plurality of droplets 106, the method can further include loading each droplet 106 of the plurality of droplets 106 into a respective microwell 126 of a plurality of microwells 126 in a microfluidic device 100. In some examples, the droplets 106 are loaded into the microwells 126 of the second structure123, or gel layer, of the microfluidic device 100. The method can further include lysing the plurality of exosomes within the plurality of droplets 106 to release a plurality of proteins from each exosome to the gel layer and applying an electric field to the gel layer. The electrophoresis machine 136 can be used to apply the electric field and conduct two-dimensional (2D) electrophoresis which was conducted in the x-y plane under separate proteins by molecular weight, and optionally followed by electrophoresis to transfer the proteins in the z-direction through the gel layer to the Raman substrate layer. In some examples, applying the electric field to the gel layer causes the plurality of proteins to migrate towards a positive electrode on the microfluidic device 100. In some examples, applying the electric field to the gel layer causes the plurality of proteins to transfer to the Raman substrate layer 129. In some examples, the method further includes detecting one or more protein signals from the plurality of proteins using Raman spectroscopy on the Raman substrate layer. In an aspect, the method can further include applying an ultraviolet (UV) light to the gel layer.

Optionally when the analysis system performed is not a Raman analysis, the proteins are not transported to the Raman substrate layer. Rather the proteins remain in the gel layer. In this embodiment, UV light can be applied by a UV light 139 to the gel lay. In some examples, applying the UV light fixes the plurality of proteins in place within the respective gel layer. Next, the method can further include adding antibodies to the gel layer. The antibodies can bind to the plurality of proteins in the gel layer. Once the antibodies have been bound to the proteins, immunoblotting can be conducted.

### EXAMPLE A

### Methodology

The present disclosure developed a new technology based on single-cell Western Blot to detect proteoform in single exosomes. Single-cell Western Blot (seWB) offers highly specific detection of proteins within cells and assesses cell-to-cell variation. The present disclosure utilizes the principles of seWB and uses droplets to encapsulate and characterize individual exosomes. The initial isolation utilizes commercially available kits to ensure specificity and yield. Subsequently, droplets are generated to encapsulate exosomes employing a similar device to seWB to allow for uniform distribution of individual exosomes (for example, see FIG. 1). Targeted immunolabelling is then used to identify specific proteins of interest, thereby increasing the sensitivity of the assay. During the detection process, fluorescent signals generated by different specific antibody-binding proteins can be used to detect the content of different proteins in individual exosomes, such as CD81, CD63, etc. Machine learning is also applied to distinguish and process the fluorescent signals. This approach refines the exosome protein analysis from average batch assessment to individual vesicle resolution, which is important for biomarker discovery and understanding of vesicle-mediated cellular processes.

This research has developed in the design and creation of a device specifically designed for single exosome analysis, based on the design, fabrication and use of silicon chips and PAGE gels in seWB. Meanwhile, the device has successfully isolated exosomes and optimized their encapsulation into microdroplets, ensuring precise control of droplet generation and subsequent loading into microwell arrays. These advances have set the stage for the next phase of research, where the aim is to complete individual exosome analysis and optimize experimental conditions to make this technique more mature.

For single exosome imaging, a high-resolution microscope system can be used to observe stained exosomes in microdroplets. This step will verify that the assay achieves the resolution of individual exosomes. By confirming the presence of individual exosomes, a robust baseline is set for further protein analysis at the level of individual vesicles.

For individual exosome protein analysis, the goal is to obtain the SERS signals of proteins in individual exosomes. The data collected can be used in a computational model that is designed to analyze and map the distribution of proteins within these vesicles. With this model, patterns and heterogeneity in protein content can be discovered, which is essential for understanding exosome function and potential diagnostic applications.

### EXAMPLE B

### Materials and Reagents

For device fabrication and single exosome detection, the following materials are employed:
SU-8 photoresist (Kayaku, 3010 or 3005)
Poly(dimethyl siloxane) (PDMS) and curing agent (Dow Corning, Sylgard 184)
Aquapel (Aquapel, cat. no. 47100)
1H,1H,2H,2H-Perfluorododecyltrichlorosilane (Sigma-Aldrich, cat. no. 729965)
FC-40 oil (3M, cat. no. 98-0212-3550-6)
2,2′-Azobis
H1299 cell line
DMEM (Mediatech, cat. no. 10-013-CV)
FBS (Life Technologies, cat. no. 10313-039)
Penicillin-streptomycin (Life Technologies, cat. no. 15140-122)
PBS (Lifetech, cat. no. 10010-023)
Trypsin (Sigma-Aldrich, cat. no. T4549)
Anti-CD45 Antibody
488 donkey anti-goat IgG
ExoQuick-TC (cat. no. EXOTC50A-1)
PKH-26 (Sigma, MIDI26-1KT)
30% Acrylamide/Bis solution 29:1 (w/v) (For polyacrylamide gels)
N,N′-Methylenebisacrylamide (BPMA)
Dimethyl sulfoxide (DMSO)
Tris-Glycine buffer
Ammonium persulfate (APS)
N,N,N′,N′-Tetramethylethylenediamine (TEMED)
Hexane
Sorbitan monooleate (Span 80)
Polyoxyethylene sorbitan monolaurate (Tween 20)

### Methodology

### Chip Fabrication

Microwell array fabrication. Microarray devices are fabricated using precise AutoCAD designs to dictate well dimensions and spacing on microarray chips. Photolithography will follow with steps involving soft baking at 95° C. for 3 minutes, exposure to SU-8 3005, and a post-bake at 65° C. for 1 minute then 95° C. for 2 minutes, culminating in a 2-minute development phase.

After photolithography, the chips (e.g., the substrate can be silanized with 1H,1H,2H,2H-perfluorododecyltrichlorosilane to create a hydrophobic surface) are prepared for gel microwell creation. Initiators APS and TEMED will be added to the gel precursor solution, taking care to avoid bubble formation. This solution will then be pipetted as a 198 μL droplet near the short edge of the SU-8 mold. A silanized slide is brought into contact with the droplet and the mold at approximately a 30° angle, then slowly lowered until it lies flat against the SU-8 mold, ensuring that the micropillars on the mold make contact with the slide. Gentle pressure is applied to the slide to extrude excess precursor and promote contact, facilitating chemical polymerization within approximately 20 minutes.

Droplet microfluidic. For droplet microfluidic chip fabrication, the process mirrors that of the microwell array up to the reverse molding in PDMS. After curing the PDMS, holes are punched for the inlets and outlet, followed by bonding the device to glass slides. Aquapel treatment ensures hydrophobicity, with a nitrogen blow dry to remove moisture before filling the channels with FC-40 oil. A final heating step at 150° C. for 1 hour reinforces device integrity.

### Single Exosome Detection Platform

Single exosome droplet generation. Exosomes are isolated using a commercial kit from the culture medium of H1299 cells. The resultant exosome suspension serves as the dispersed phase, which, along with the VA-086 gel precursors, generates 7 μm droplets. The continuous phase, consisting of mineral oil with Span 80 (e.g., 2 % 008-FluoroSurfactant in HFE7500 (Ran Biotechnology)), facilitates the formation of droplets at controlled flow ratios, which are solidified through UV polymerization and washed in hexane with Span 80 followed by PBS.

Microwell array loading. The exosome-loaded droplets are then introduced to the microwell array. Post-loading, the microarray undergoes air plasma treatment and centrifugation to ensure proper exosome placement within each well, followed by PBS washes to prepare for proteoform detection.

### Proteoform Detection Using SERS

AgNR substrate preparation. The AgNR substrate preparation begins with the fabrication of silver nanorod arrays using the oblique angle deposition (OAD) method in an electron-beam evaporation system on glass slides. After the AgNR arrays are formed, a SiO2 coating is used to enhance stability and biocompatibility. To start, the AgNR arrays are rinsed with deionized water and dried with nitrogen, then immersed in a homogeneous mixture consisting of 30 mL of ethanol (EtOH), 4 mL of deionized water, and 500 μL of TEOS. To initiate the coating reaction, 560 μL of ammonium hydroxide (28.0-30.0 wt%) is added to the TEOS mixture. The coating thickness is controlled by adjusting the hydrolysis time of the TEOS.

Diffusion-based Protein Detection. 5 μL of a pure protein solution is added onto the surface of the gel, appropriately sized based on the gel's dimensions. The solution is spread by scraping it slightly with a coverslip to ensure even distribution. After the solution has dried naturally, the substrate is placed under a microscope, ensuring the microwells are visible. The measurements begin by directing a laser into a microwell for ‘in well’ measurements, and also take measurements outside the microwell for ‘out of well’ comparisons, recording all data accordingly.

Electrophoresis-based Protein Detection. 5 μL of a pure protein solution is added onto the surface of the gel, appropriately sized based on the gel's dimensions. An electric field is applied in the x-y plane to facilitate the separation of proteins, z-direction electric field is then applied, promoting the vertical migration of the separated proteins through the PAGE gel towards the SERS substrate. Post-electrophoresis, the gel is gently moistened to aid in its removal from the substrate and the spectra can be collected by the substrate.

### Signal Processing and Statistical Analysis

The gathered SERS signals undergo robust processing to extract quantitative data, which is then statistically analyzed to validate the experimental findings, ensuring the detection platform's efficacy is objectively assessed.

### Results

Microwell Array and Microfluidic Device. The platform consists of a microwell array and microfluidic device designed for single exosome separations and analyses, respectively.

The microwell array consists of wells with a diameter of 7 μm, a gap length of 200 μm, and a width of 40 μm, optimizing the spatial arrangement for efficient PAGE-gel formation. In the microfluidic component, a T-junction droplet microfluidic device is utilized to generate water-in-oil (W/O) droplets at the outlet. The device features two continuous-phase inlets: one controlling the buffer solution and the other managing the exosome suspension. The disperse-phase inlet introduces oil (FC-40) with surfactant, ensuring the stabilization of droplets within the system.

Exosome Isolation and Staining. Exosomes are isolated using the EXO-Quick commercial kit from 2 types of cancer cell lines (H1299, MDB-MCF7), a widely recognized and efficient method for isolating exosomes from various biological fluids. This kit utilizes a polymer-based precipitation technique, which allows for the quick and reliable recovery of intact exosomes. Following isolation, the exosomes are stained using PKH-26, a fluorescent dye that binds to the lipid membrane of vesicles.

Single Exosome Droplet Generation. The droplet generation process is carried out using an 8 μm droplet device. The droplets are produced in a water-in-oil (W/O) emulsion system, utilizing a 0.45 μm filter with a dispersed phase to continuous phase flow ratio of 2 μL/min to 4 μL/min. The size of the generated droplets is approximately 11.57 μm. One of the inlets of dispersed phase consists of 0.5% (w/v) VA-086, 8% (v/v) acrylamide/bis-acrylamide, 1X Tris-Glycine, and water, along with another inlet of the exosome suspension solution. The continuous phase is composed of mineral oil containing 2% (v/v) Span 80 (2 % 008-FluoroSurfactant in HFE7500 (Ran Biotechnology)). The flow ratio for this phase ranges from 0.6-2 μL/min to 5-2 μL/min, yielding droplets with a size of around 11.9 μm. The droplets are polymerized under UV light for 60 seconds.

Single Exosome Seeding. Prior to seeding, the droplets undergo a washing step to remove excess oil and surfactant. Using hexane with 1% (v/v) Span 80, followed by centrifugation at 2500 g for 5 minutes. The water-in-oil emulsion was broken by adding 20% perfluorooctanol (PFO) in FC-40 oil to destabilize the oil phase and release the droplets. The droplets were collected and washed three times with hexane containing 1 % (v/v) Span 80, followed by centrifugation at 2500 g for 5 minutes to remove excess oil and surfactant residues. After centrifugation, the droplets are washed with PBS to ensure the removal of any remaining impurities. Then the generated droplets are seeded into a microwell array on a PAGE gel.

### Single Exosome Proteoform Analysis

Simulation and Rhodamine b Test. In the simulation comparing diffusion and electrophoresis, Bovine Serum Albumin (BSA) exhibited distinct differences in behavior under each condition within the gel matrix. In diffusion-only scenarios, BSA movement process was relatively slow based on molecules spread from areas of high concentration to low concentration over time. In contrast, applying an electric field during electrophoresis significantly accelerated BSA migration through the gel.

The Rhodamine B test results provided insights into the behavior of Rhodamine B within the PAGE-gel system, especially regarding its diffusion and interaction with the gel matrix. When measuring the diffusion process, significant peaks were observed at concentrations higher than 10-4 mg/mL, corresponding to the known vibrational modes of Rhodamine B. However, at lower concentrations, the Raman signal became irregular.

During the time-relevant assay, signal intensity decreased slightly after 60 minutes, suggesting that moisture levels and sample diffusion affected the measurement. Mapping experiments demonstrated that Rhodamine B signals in microwells were more intense compared to those outside, supporting the hypothesis that the thinner gel layer in microwells enhanced signal intensity. Additionally, comparing Rhodamine B measurements in PAGE-gel with those after gel removal indicated that removing the gel significantly improved signal intensity.

A comparison of Rhodamine B signals within and outside of microwells using a 633 nm laser revealed that the signal inside microwells was more stable, whereas outside the microwells, oscillations occurred due to the membrane effects.

A laser comparison between 785 nm and 633 nm showed that the 633 nm laser provided much stronger and more sensitive detection of Rhodamine B, especially at concentrations below 10-4 mg/mL. The signal intensity with the 633 nm laser was about twice that of the 785 nm laser, indicating its suitability for low-concentration detection in similar experiments.

Pure protein SERS spectra. BSA488, which is Bovine Serum Albumin conjugated with Alexa Fluor 488, and OVA555, which is ovalbumin conjugated with Alexa Fluor 555, were analyzed using SERS after being diluted to 1mg/mL in RIPA buffer with 2% SDS. The SDS and RIPA not only helped to solubilize the proteins but also ensured they carried a consistent negative charge, which is essential for accurate protein migration during electrophoresis.

Electrophoresis-based protein signal processing. Electrophoresis was applied to process the protein signals, with a 1 V electric field applied for 60 seconds in the z-direction across a PAGE-gel with a thickness of 100 μm.

### EXAMPLE C

Proteoforms are specific molecular forms of a protein, encompassing all the protein's variations due to genetic differences, alternative splicing, and post-translational modifications. Identification of proteoforms within a single cell is an important process that can lead to discoveries in cell function and disease progression. Standard western blotting identifies proteins present within a large group of cells but cannot identify protein content at the single cell level. Techniques for single cell western blotting (seWB) have been developed but the immunoprobing step is time-intensive, depends on known antibodies, and requires a large amount of protein for detection. To remedy the issues with immunoprobing, the present disclosure is a new technique that combines methods from seWB and surface enhanced Raman spectroscopy (SERS), a powerful analytical technique that detects and identifies molecules at low concentrations, down to a single molecule. When paired with seWB this could yield an improved method for identifying proteoforms within a single cell. First, the feasibility of seWB with SERS was tested to ensure the new technique can be implemented and improve upon current technologies' shortcomings. Rhodamine B (RhoB) was used as a test target. The initial step was to determine if RhoB can be detected by SERS after diffusing through polyacrylamide gel (PA-gel) onto the SERS substrate. Then, the optimal time to perform SERS was found after protein loading and passive diffusion. Finally, COMSOL was used to simulate and compare passive diffusion and 3D gel electrophoresis as methods for protein travel down to the SERS substrate.

### Materials and Methods

Device fabrication. Wafer microfabrication and silanization were conducted based on established protocol. Each microwell had a diameter of 30 μm and a depth of 40 μm. An 8% PA-gel was prepared by mixing 30% (w/w) acrylamide/bis-acrylamide, gel strengthener, 10× Tris-glycine buffer, and ddH2O and was polymerized for 40 minutes using 0.08% (w/v) APS and 0.08% (v/v) TEMED. Adhesive tape is used as a spacer to define the gel boundary and achieve a uniform thickness of 100 μm. After polymerization, gels were delaminated from the wafer using a razor blade and stored in DI water. Silver nanorods (AgNR) were deposited onto a glass slide and plasma cleaned with argon for SERS analysis.

Sample preparation. PA-gel was placed onto an AgNR substrate. RhoB (0.01 mg/mL) was pipetted directly onto the PA-gel. A second sample was prepared by directly pipetting RhoB onto AgNR substrate and then covering with PA-gel. Spectra were collected in and out of the microwells. For each sample 9 SERS spectra were collected with Renishaw inVia confocal Raman microscope and WiRE software using 785 nm laser. OriginPro was used to average spectra and remove the baseline using asymmetric least squared smoothing.

For the second experiment, PA-gel was placed onto AgNR substrate, RhoB (0.01 mg/mL) was pipetted onto PA-gel, and SERS spectra were collected after 10, 15, 20, 45, 60, and 100 minutes of diffusion. The spectra were analyzed for the intensity of 5 different RhoB peaks.

Simulation. BSA was simulated to be moving downwards from a 40 μm diameter microwell. First, passive diffusion was simulated over 60 minutes. Second, 3D gel electrophoresis was simulated over 5 seconds.

### Results and Conclusions

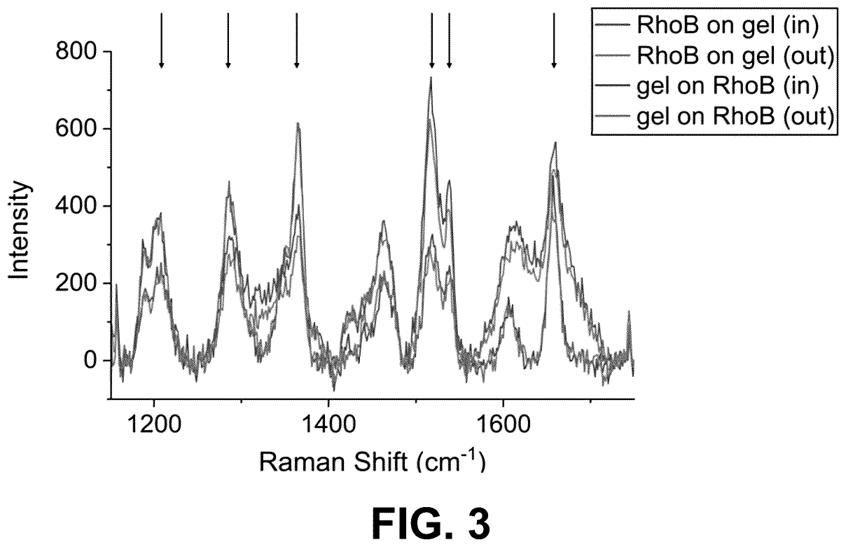

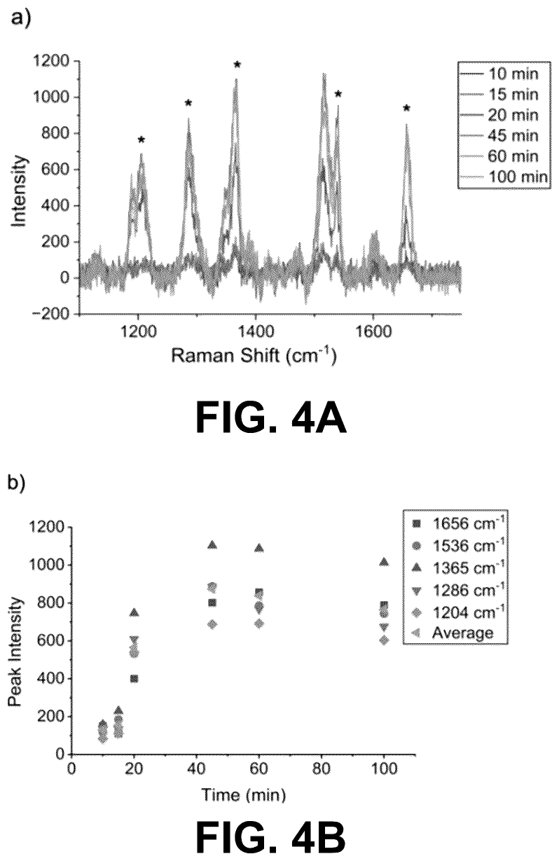

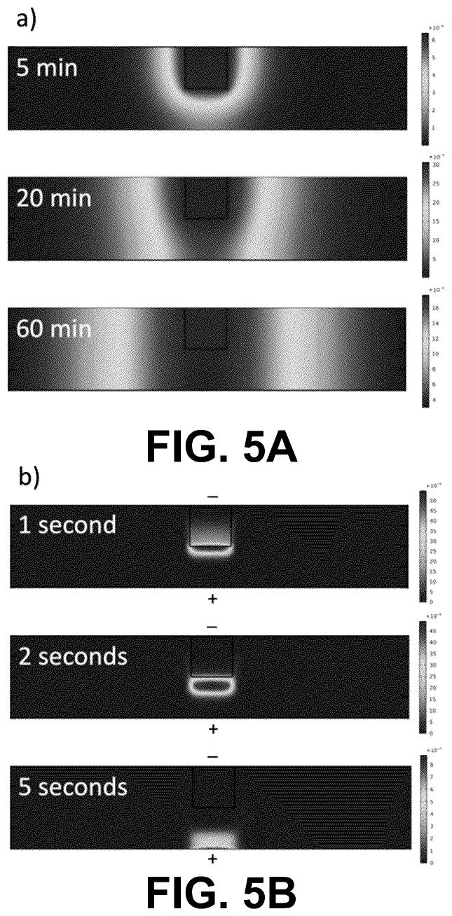

The results support the feasibility of using SERS as an effective replacement for immunoprobing in seWB. When comparing loading RhoB on top of the gel against loading RhoB directly on the substrate and covered by the gel, all four spectra contain the same signature peaks for RhoB (see FIG. 3). Despite differences in intensity between the two loading methods, both give rise to all the signature peaks and can detect the protein while imaging through the PA-gel. The optimal time point to collect data when using passive diffusion of RhoB through the gel was found to be around 45 minutes (see FIGS. 4A-B). After 45 minutes, the peak intensity begins to decrease which may be due to excess diffusion away from the substrate. Finally, the simulation suggests that gel electrophoresis will be a successful method of transferring proteins from the microwells down to the AgNR substrate (see FIGS. 5A-B). The simulation predicts that 3D electrophoresis will result in 50 times more BSA located on the AgNR substrate than relying solely on passive diffusion, providing much higher protein detection.

This data shows the feasibility of single cell western blotting coupled with surface enhanced Raman spectroscopy to identify the protein contents of a single cell. Since the detection of RhoB is possible, the next steps include repeating these experiments with other proteins of different molecular weights (e.g., Bovine Serum Albumin, Immunoglobin, Ovalbumin). Additionally, identification of protein molecules at low concentrations should be further investigated and a physical set up for electrotransfer of proteins onto the AgNR should be developed.

Disjunctive language such as the phrase “at least one of X, Y, or Z,” unless specifically stated otherwise, is otherwise understood with the context as used in general to present that an item, term, etc., can be either X, Y, or Z, or any combination thereof (e.g., X; Y; Z; X or Y; X or Z; Y or Z; X, Y, or Z; etc.). Thus, such disjunctive language is not generally intended to, and should not, imply that certain embodiments require at least one of X, at least one of Y, or at least one of Z to each be present.

It should be emphasized that the above-described embodiments of the present disclosure are merely possible examples of implementations set forth for a clear understanding of the principles of the disclosure. Many variations and modifications can be made to the above-described embodiments without departing substantially from the spirit and principles of the disclosure. All such modifications and variations are intended to be included herein within the scope of this disclosure and protected by the following claims.

Therefore, the following is claimed:

1. A microfluidic system, comprising:
a first structure including a first channel configured to receive a plurality of exosomes, a second channel configured to receive a hydrogel precursor, where the second channel merges with the first channel to form a third channel, wherein the exosomes are within the hydrogel precursor in the third channel, and a fourth channel intersecting the third channel, wherein the system is configured to form a plurality of droplets in the third channel after the intersection with the fourth channel; and
a second structure having a first plurality of microwells, wherein each microwell is configured to receive a single droplet, wherein each microwell includes a top layer, wherein the top layer is a gel layer having a top side and a bottom side, wherein the gel layer receives the droplet on the top side.

2. The microfluidic device of claim 1, further comprising a bottom layer in the second structure comprising a Raman substrate layer, wherein the Raman substrate layer is adjacent the bottom side of the gel layer.

3. The microfluidic device of claim 1, wherein each microwell of the first plurality of microwells has a diameter of about 1.5 to 15 μm and a depth of about 2 to 20 μm.

4. The microfluidic device of claim 1, wherein the fourth channel is configured to cross-flow a fluid against the plurality of exosomes in the hydrogel precursor to form the droplet.

5. A method, comprising:
loading a plurality of exosomes into a respective plurality of droplets, such that each exosome of the plurality of exosomes is contained within a respective droplet of the plurality of droplets;
loading each droplet of the plurality of droplets into a respective microwell of a plurality of microwells in a microfluidic device;
lysing the plurality of exosomes within the plurality of droplets; and
applying an electric field to release a plurality of proteins from each exosome to a gel layer.

6. The method of claim 5, where the applied electric field comprises about 10-40 V/cm on x-y plane electrophoresis, and optionally the applied electric field comprises about 1-10 V/cm on z-direction electrophoresis.

7. The method of claim 5, wherein applying the electric field to the gel layer causes the plurality of proteins to migrate towards a positive electrode on the microfluidic device.

8. The method of claim 5, further comprising applying an ultraviolet (UV) light to the gel layer.

9. The method of claim 8, wherein applying the UV light fixes the plurality of proteins in place within the gel layer.

10. The method of claim 8, further comprising adding antibodies to the gel layer after applying the electric field.

11. The method of claim 10, wherein the antibodies bind to the plurality of proteins in the gel layer.

12. The method of claim 5, further comprising detecting the plurality of proteins using SERS.

13. The method of claim 5, further comprising detecting the plurality of proteins using immunolabeling.

14. The method of claim 5, wherein loading the plurality of exosomes into the plurality of droplets further comprises:
inserting one exosome into a hydrogel precursor;
flowing the hydrogel precursor and plurality of exosomes into a microchannel; and
intersecting the flowing of the hydrogel precursor with a crossflow of oil to form the droplets.

15. The method of claim 5, further comprising transferring the plurality of proteins from the gel layer to a Raman substrate layer of the microfluidic device by conducting electrophoresis.

16. The method of claim 15, further comprising detecting one or more protein signals from the plurality of proteins using Raman spectroscopy.

17. A system, comprising:
a microfluidic device having a first structure and a second structure,
wherein the first structure comprises: a first structure having a first channel configured to receive a plurality of exosomes, a second channel configured to receive a hydrogel precursor, where the second channel merges with the first channel to form a third channel, wherein the exosomes are within the hydrogel precursor in the third channel, and a fourth channel intersecting the third channel configured to cross-flow a fluid against the plurality of exosomes in the hydrogel precursor to form the droplet, wherein the third channel is in fluidic communication with the plurality of microwells;
wherein the second structure includes a plurality of microwells, each microwell of the plurality of microwells configured to receive a single droplet; and
an electrophoresis machine configured to apply an electric current to the plurality of microwells.

18. The system of claim 17, wherein each microwell of the plurality of microwells contains a droplet, the droplet encasing a single exosome.

19. The system of claim 17, wherein each microwell of the first plurality of microwells has a diameter of about 1.5 to 15 μm and a depth of about 2 to 20 μm and wherein microfluidic device has 5,000-500,000 microwells, wherein the microwell spacing in the y-direction is about 100 μm×1000 μm, wherein the microwell spacing in the x-direction is about 20 μm×200 μm.

20. The system of claim 17, further comprising a Raman spectroscopy device.
